(19) United States
(12) Patent Application Publication
(10) Pub. No.: US 2009/0227003 A1
(43) Pub. Date: Sep. 10, 2009

(54) Methods and Systems for Biomass Recycling and Energy Production

(76) Inventors: Roger Blotsky, Goodyear, AZ (US); Gary Fred Stepenoff, Phoenix, AZ (US); Ramon Figueroa, Miami, FL (US); Gary Scott Stepenoff, Phoenix, AZ (US)

Correspondence Address: Ballard Spahr Andrews & Ingersoll, LLP, SUITE 1000, 999 PEACHTREE STREET, ATLANTA, GA, 30309-3915, US

(21) Appl. No.: 12/341,759

(22) Filed: Dec. 22, 2008

Related U.S. Application Data
(60) Provisional application No. 61/008,704, filed on Dec. 21, 2007.

(51) Int. Cl. C12N 1/12; C02F 11/04; C12M 1/107

(52) U.S. Cl. 435/257.1; 435/300.1; 210/603

(57) ABSTRACT

The present invention comprises methods and systems for treating biomass wastes to result in usable byproducts. Biomass is treated to remove debris, transferred to microbial digester units, such as anaerobic and aerobic digesters, and the resultant solids and liquids are provided to an algae production unit. Algae are harvested and beneficial byproducts are retained. Gases, heat and energy produced by energy conversion units are used in units of the system or provided to external sources. Water is cleaned and when separated from the algae and other solids in the algae harvesting unit may be provided to external sources, or may be used in other units of the system. The methods and systems disclosed herein provide for an efficient and substantially complete use of the components of the input biomass.

### RELATED APPLICATION

This application claims the priority of U.S. Provisional Patent Application 61/008,704, filed Dec. 21, 2007, which is herein incorporated in its entirety.

### TECHNICAL FIELD

The present invention relates to methods, devices and systems for recycling animal or plant biomass waste materials and to produce energy and beneficial products therefrom.

### BACKGROUND OF THE INVENTION

Waste materials from agriculture and humans, biomass, have long been used for multiple functions. Many of these functions require reconditioning the wastes to yield new products and other resulting outcomes, such as energy and heat. Microorganisms have been used in many reconditioning systems to alter the biomass, as have mechanical and chemical means.

The extraction of products and production of energy from biomass wastes using anaerobic digestion and the general technology of digester devices is known. Anaerobic digestion has been used to treat sewage sludge, and often the resulting biogas has been used to maintain the digester temperature or to run internal combustion engines. Small units have been used to produce gas for homes and farms for everyday activities such as cooking or hot water heating.

Anaerobic digestion is a microbial process involving biochemical decomposition of organic material such as animal manures in the absence of oxygen. It is a two-stage process where microorganisms convert the complex organic matter present in biomass into simple organic acids that then are acted on to produce methane, nitrogen containing gases, and carbon dioxide gases. A digester device is used to control the nutrient loading rate, retention time, mixing and temperature for optimum production of the gases.

Numerous aerobic processes have been developed over the years for the biological treatment of biomass such as municipal waste which includes both domestic and industrial sewage to yield an environmentally acceptable effluent. One aerobic process is the activated sludge process where organic matter contained in municipal waste is contacted with an oxygen-containing gas in the presence of suspended biologically active organisms under conditions such that the organic material is converted into a form which can be separated from purified water. A portion of the insoluble sludge that is formed may be recycled to the aerobic zone. Another method of aerobic treatment is a trickling filtration method wherein the microorganisms are fixed to a support.

In some instances, some of the byproducts of these biomass treatment processes have been retained and used in the system, such as heat recycling, but systems for efficiently using the input biomass to produce energy, heat and byproducts that can be used in the system itself and/or provided to external sources are not found.

Additionally, problems exist within the biomass treatment systems as they exist. In aerobic systems, there is usually a significant net positive production of sludge containing suspended solids in the process and there is an increasing inventory of sludge. Excess sludge must be discarded on a periodic basis from the process. Biological sludges produced by the activated sludge process and other aerobic processes are difficult and expensive to treat because these sludges have poor dewatering properties and are highly putrescible. Because of these characteristics, sludge disposition has become an issue. Ocean dumping of sludge or use as landfill are objectionable because such sludges present health hazards to the environment. Prior to any type of disposal, these sludges require pasteurization so that the concentration of pathogenic organisms is sufficiently low to avoid potential health hazards. An issue with anaerobic digestion is its high cost in operation and substantial time is required for the digestion process and the equipment tends to be of a large scale.

What is needed are systems and methods for treatment of biomass materials that reduce the amount of residual material that must be disposed of, that extracts the energy stored in the biomass, and that produces beneficial byproducts using the components of the biomass waste.

### SUMMARY

The present invention comprises methods and systems for the recycling of the nutrients, compounds and energy of biomass waste materials into usable gases, electricity, heat, cleaner water and beneficial products such as protein sources, fertilizer, biodiesel, pharmaceutical agents, and others. The present invention comprises treating biomass materials using microbial digester units and/or algae production units. The methods, systems and units of the present invention may be interrelated so as to use byproducts produced in one or more units or method steps of the invention.

An exemplary method and system of the present invention comprises the following. A biomass is provided, which may be agricultural, animal or human waste material, and the biomass may be pretreated by a debris separation unit. In the debris separation unit, debris is removed from the biomass. The biomass feedstock is transferred to a second unit, such as one or more hydrolysis units or one or more microbial digester units, such as an anaerobic digester unit and/or an aerobic digester unit. In an hydrolysis unit, the biomass may release carbon dioxide or other gases which may be captured and used in other units in the methods and systems, stored, provided to external recipients, or released. In an anaerobic digester, the biomass is held in an anaerobic environment and the biomass feedstock is acted on by anaerobic microorganisms to result in gases and treated biomass liquids and solids.

From an anaerobic digester, the output, one or both of the solids and liquids of the treated biomass, may be transferred to an aerobic digester unit where the treated biomass solids and/or liquids are then held in an aerobic environment so that microorganisms act on the biomass material. From an anaerobic digester unit, the output may be transferred to an algae production unit. After treatment in an aerobic digester unit, the output, aerobically treated solids and liquids of the biomass, may be transferred to an algae production unit. In an algae production unit, the treated biomass is utilized to grow algae. The algae may be harvested for multiple uses.

Gases produced by an hydrolysis unit, an anaerobic digester, an aerobic digester and/or an algae production unit may be removed and separated by a gas separation unit, or provided directly to one or more units in the system, to external recipients or stored. For example, methane from an anaerobic digester may be isolated from a mixture provided to a gas separation unit and is transferred to an energy generation unit and burned to produce electricity, heat and carbon dioxide. Carbon dioxide from an energy generation unit, and/or from an aerobic digester and/or an hydrolysis unit may be provided to an algae production unit. Nitrogen containing gases, such as ammonia, (NH₃), may be separated by a gas separation unit from other gases produced in an anaerobic digester and the gases may be stored or used for other purposes. For example, nitrogen containing gases can be used to supply nitrogen to an algae production unit to enhance algal growth.

Heat from an energy production unit may be provided to one or more other units in the methods and the systems herein, such as an anaerobic digester, an algae production unit and/or an aerobic digester, or provided to external sites, or both.

Electricity produced at an energy generation unit may be provided to one or more other units in the system or to external recipients, or both.

Water separated from an algae and other solids in the algae harvesting unit may be provided to external recipients, or may be used in one or more other units of the system, such as adding to the input biomass prior to debris separation.

### DESCRIPTION OF FIGURES

FIG. 1 shows an exemplary embodiment of a method and system of the present invention.

FIG. 2 shows an exemplary embodiment of a method and system of the present invention.

### DETAILED DESCRIPTION

The present invention comprises methods and systems for treating biomass, recycling and creating useful results therefrom, including heat, energy and beneficial products. In general, methods of the present invention comprise use of one or more units to treat a biomass to yield products, including but not limited to, gases, algae, liquids, and solids. An example of treatment of a biomass comprises, preparation of the incoming biomass feedstock to remove unwanted materials, optionally, retention of the biomass feedstock in an hydrolysis unit, anaerobic digestion of the biomass feedstock, aerobic digestion of the liquids and solids derived from an anaerobic digester, providing the liquids and solids from an aerobic digester to an algae production unit, and harvesting the algae, for use of the algae or further processing of the algae to produce beneficial products. Methods comprise use of gases produced from an hydrolysis unit, an anaerobic digester or an aerobic digester in an energy generation unit, or providing such gases in other method steps or system units. Methods comprise use of heat produced at one or more steps in the method or units in the system, such as from an energy generation unit or an anaerobic and/or aerobic digester in other method steps such as a microbial digester unit or an algae production unit. Methods comprise recycling or reuse of water from one unit or step for use in other steps or system units, or providing water cleaned by a system of the present invention to agricultural, recreational or municipal water systems.

Systems of the present invention comprise one or more units for preparation of an incoming biomass to remove unwanted materials, units for retention of the biomass, units for anaerobic digestion of the feedstock, units for aerobic digestion, conduits for transporting the liquid and solid resultants to and from other units in the system, and units for algae production and or use of the algae or units for further processing of the algae to produce beneficial products. Systems comprise use of gas transfer conduits and units for gas processing of gases produced from units such as anaerobic or aerobic digester units, an energy generation units, or for providing such gases in other system units or components. Systems comprise heat transfer conduits and units for heat transfer and processing for the use of heat produced by differing units in the system, such as from the energy generation unit or the digester units to other units such as the digester units or the algae production unit. Systems comprise conduits and processing units for recycling or reuse of water from one unit for use in other units, or units for providing cleaned water to agricultural, recreational or municipal water systems.

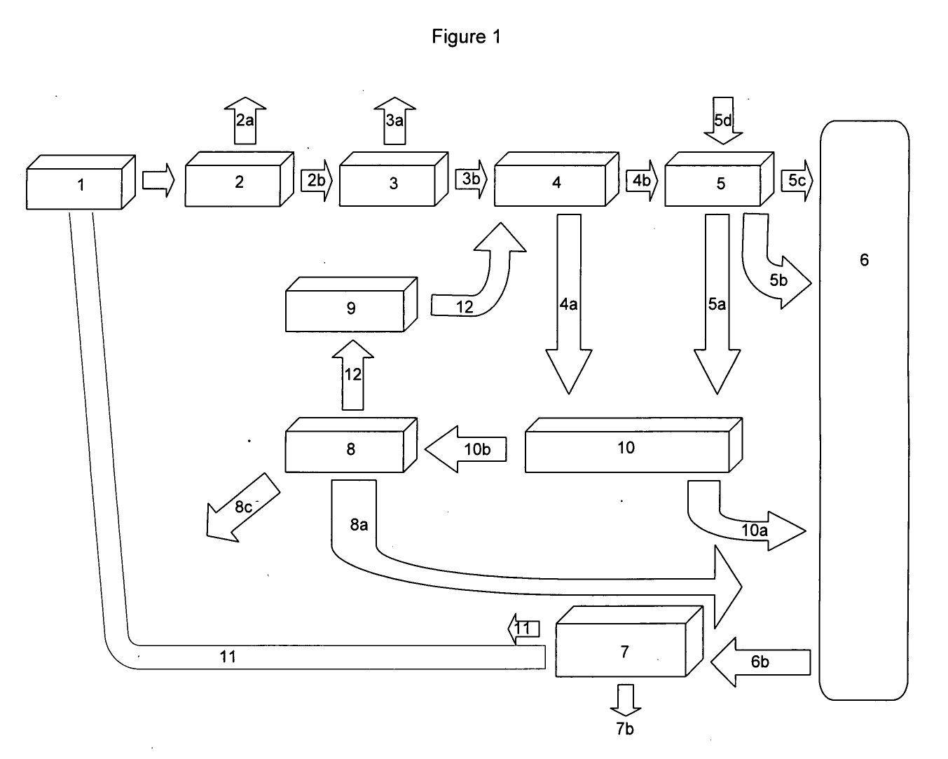

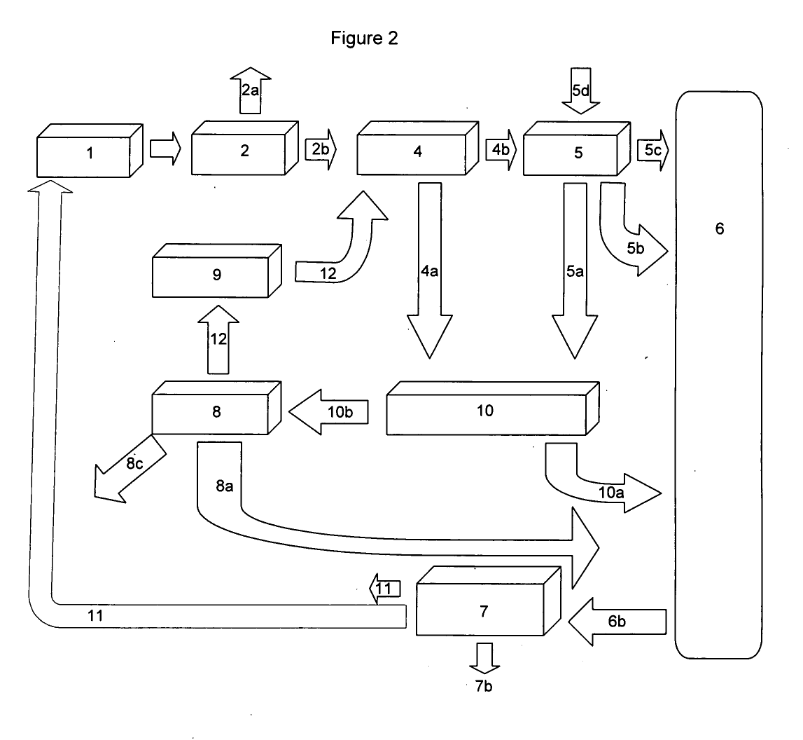

The present invention comprises methods and systems for deriving energy from biomass and treating the biomass to recycle its components and to produce beneficial products. In general, an aspect of the present invention comprises the conversion of animal and/or plant waste material to produce energy and other beneficial products by use of microbial digestion methods, conversion of byproduct gases to electrical energy via combustion destruction of methane and other gases. Methods and systems comprise utilization of produced carbon dioxide for growing algae, including but not limited to, diatoms, which aids in the reduction of greenhouse gases, such as methane and carbon dioxide emissions. Processing of the algae may result in a variety of products including biodiesel fuel, oils, lipids and fatty acids, protein for use in animal and human food, enzymes and alcohols for industrial uses and other beneficial products and uses. FIGS. 1 and 2 show embodiments of methods and systems of the present invention. Beneficial products and uses include, but are not limited to methane gas, hydrogen gas, ammonia gas, carbon dioxide, hydrogen sulfide, nitrogen rich fertilizer, proteins, amino acids, carbohydrate and/or mineral rich compositions, solutions or slurries, insecticidal mixture, charcoal, carbon black, insect repellant mixture, biodiesel, algae, algae products, heat, electricity, potable water, grey water, and combinations thereof and other such materials which can be prepared from a biomass feedstock.

The methods and systems of the present invention recycle the energy, nutrients, water and minerals contained in biomass material, including, but not limited to, manure, agricultural wastes and human wastes. The methods and systems utilize the energy, nutrients, and water in the production of electrical energy, the capture of heat in a usable form, and the production of plant oils, proteins and carbohydrates. Methods and systems will reduce the methane and carbon dioxide currently being released into the atmosphere from these biomass waste sources. The methods and systems capture and utilize the nitrogen, potassium, phosphorous present in the biomass material for production of proteins, oils and carbohydrates by algae or other plant sources, and thus reduce the discharge of liquid wastes that contribute to groundwater contamination. Water present in the biomass waste feedstock and used in the methods and systems is cleaned of high levels of nutrients which allows for the water to be used for agricultural and municipal purposes.

In the methods and systems of the present invention, input materials are provided to units and output materials result from the actions of the units. Output materials may be stored, used in the unit wherein it was produced or other units of the system, or transferred for external uses. Input material may be pretreated before providing to a unit of the present invention.

An example of methods and systems of the present invention comprise one or more of the following steps or units. Input material, such as biomass, is provided to a debris separation unit. Output material from a debris separation unit comprises a biomass feedstock and indigestible materials separated from the original biomass. Input material, such as a biomass feedstock, may be provided to an hydrolysis unit, an anaerobic digester, an aerobic digester, or an algae production unit. In an embodiment, the biomass feedstock is provided to an hydrolysis unit, where the biomass feedstock is retained for a specific period of time, and is then transported to an anaerobic digester unit. In an embodiment, the biomass feedstock is provided to an anaerobic digester unit. In an embodiment, the biomass feedstock is provided to an aerobic digester unit. In an embodiment, the biomass feedstock is provided to an algae production unit. An used herein, the singular form of a unit is used, such as “an aerobic digester unit” and this term encompasses the use of one or more of these units being used.

Output material of an anaerobic digester comprises solids and liquids resulting from anaerobic activity with the biomass feedstock, and gases produced during the anaerobic process. Such gases include, but are not limited to, hydrogen, methane, ammonia, hydrogen sulfide, carbon dioxide. Such gases may be used in other units of the methods and systems of the present invention, stored or provided to external recipients.

Input material is provided to an aerobic digester unit, and may comprise output material from an hydrolysis unit, output material from an anaerobic digester unit, a biomass feedstock, or other materials capable of being acted on by an aerobic digester unit. Aerobic digester unit output material includes solids and liquids that result from the actions of the microorganisms or organisms in the aerobic digester unit, and gases. Input material is provided to an algae production unit and may comprise output material from an hydrolysis unit, an aerobic digester unit, a biomass feedstock, or other materials capable of being acted on by the algae, organisms and microorganisms present in the algae production unit. Output material from the algae production unit comprises solids and liquids resulting from the action of the organisms and/or microorganisms of the algae production unit, algae and gases. Input material is provided to a gas separation unit and output material includes separated gases. Input material, such as a gas mixture, is provided to an energy production unit and output material comprises energy, heat and gas, such as carbon dioxide.

Methods of the present invention comprise use of plant and animal biomass as a feedstock for the methods and systems described herein. As used herein, the terms “biomass” and “biomass feedstock” and “feedstock” are taken to mean any animal or plant derived material that contains one or more components that can be converted, bioconverted or biodegraded into a useful material by the methods and systems of the present invention. Biomass comprises, but is not limited to, biological waste such as fecal waste from agricultural sources including but not limited to dairy cattle, beef cattle, poultry, such as chickens, turkeys, ducks, and swine, collected in the day to day production of animal husbandry operations; animal tissue and/or biomass, fish tissue or parts, plant parts, fruits, vegetables, plant processing waste, animal processing waste, animal manure or urine, mammalian or human manure or urine solids isolated from fermentation cultures, and combinations thereof are included in the term feedstock. Particular examples of biomass include bovine, poultry, equine or porcine manure or urine, wood shavings or chips, slops, shredded paper, cotton burrs, grain, chaff, seed shells, hay, alfalfa, grasses, leaves, sea shells, seed pods, corn shucks, weeds, aquatic plants, algae and fungus and combinations thereof.

The biomass may be pretreated. A pretreatment step comprises removal of nondigestible material such as concrete or undigestible debris in a debris separation unit. The nondigestible material may be removed by in a debris separation unit comprising sieves, screens, density flotation devices or materials, or a debris separation unit comprising hydrocyclone devices for removing debris. Such hydroclone devices are taught in U.S. Pat. Nos. 5,284,250 and 6,973,980, each of which is herein incorporated in its entirety. A unit for removing debris, a debris separation unit (DSU) is used to pretreat the biomass prior to provision of the biomass feedstock to another unit of the system, such as an anaerobic digester unit. Depending on the source and condition of the biomass, it may be screened, separated and/or otherwise processed to remove undesirable foreign matter, such as nondigestible materials, from it. This pretreatment removes debris which may have been loaded or mixed with the biomass during the scraping, loading, hauling, or unloading stages of transportation of the feedstock. By separating the nondigestible material the overall volume of the starting material, including biomass and nondigestible material, will be reduced. Oversized debris and foreign materials (sand, excessive clays, plastics, wood, rocks, concrete, clothing,) are sequestered into a separate container for disposal, such as in a landfill, or for return to the source of the feedstock, such as a farm or feedlot.

The particle size of the biomass can be reduced either prior to or during removal of debris by employing an in-line or immersed abrader, classifier, mill, high shear mixer, grinder, homogenizer or other particle size reducer known to those of ordinary skill in the art. No particular particle size is required for the biomass; however, smaller particle sizes may be bioconverted more quickly than larger particles. Grit, such as dirt, sand, soil, stones, pebbles, rocks, feathers, hair and other such materials, may be removed at this time or at any point in the system or methods. Equipment such as classifiers, settling tanks, multiphase tanks, and/or or filters can be used to remove the grit.

After debris removal, the moisture level of the biomass feedstock is determined, and if necessary, liquid may be added to create a biomass feedstock form that is convenient for transport, storage, conveyance, pumping, digestion and separation into subcomponents. For example, a biomass feedstock slurry may be prepared by suspending a feedstock in an aqueous solution to form a slurry comprising 90% wt solids, less than about 90% wt. solids, about 0.1-60% wt. solids, or about 1-40% wt. solids, depending on the desired concentration of feedstock.

The added liquid may be water, or water containing other materials, including materials that would aid in transport, pumping, storage, digestion or separation. Water from sources such as municipal water systems, wells, surface ponds, irrigation canals, lakes or other sources may be used or the water may be recycled from other steps or units in the methods and systems of the present invention. For example, the input water may come from a dewatering step and/or harvesting step of the algae production unit. Once the biomass feedstock is determined to have an appropriate level of moisture, it is then conveyed by appropriate means, including waste pump, trash pump, disc pump, screw conveyor, peristaltic pump, mechanical loader or other transfer units or methods. For example, the moistened biomass feedstock or biomass feedstock not moistened is conveyed to at least one anaerobic digester unit for digestion and recovery of components.

Optionally, the moistened biomass feedstock or biomass feedstock not moistened is conveyed to at least one hydrolysis unit for retention of the biomass feedstock. Retention of the biomass feedstock in an hydrolysis unit allows for the capture of carbon dioxide, or other gases released by the biomass. The gases may be provided to other units of the system. The retention of the biomass may allow for transition of facultative or other anaerobes present in the biomass feedstock to convert to an active anaerobic state, or to increase in number.

An hydrolysis unit is a container, such as a closed tank, having at least one inlet and at least one outlet for providing the biomass feedstock into and out of the hydrolysis unit. The container may be open to the exterior environment, should the capture of gas not be desired. The temperature of the hydrolysis unit may or may not be controlled. Generally, a biomass feedstock is retained within the hydrolysis unit for about 2 hours to about 48 hours, for about 8 hours to about 36 hours, for about 12 to about 24 hours, for about 12 to about 18 hours. During this retention time in the hydrolysis unit, the biomass may be stationary, or may be stirred. Debris associated with the biomass feedstock may settle out and be removed from the hydrolysis unit. Water or other liquids may be added to the biomass feedstock in the hydrolysis unit. It is contemplated by the present invention that treatment of the biomass feedstock in one or more hydrolysis units may decrease the time the biomass feedstock resides in an anaerobic digester unit, and increase the efficiency of an anaerobic digester unit. The biomass feedstock may be provided directly to a microbial digester unit, anaerobic or aerobic, or may be provided to an hydrolysis unit prior to being provided to a microbial digester unit. For example, after retention in the hydrolysis unit, the resultant liquids and/or solids are provided to an anaerobic digester unit.

A step in the treatment of the biomass feedstock is anaerobic digestion. One or more anaerobic digester units may be employed in the methods and systems of the present invention, and if more than one is used, may be used in parallel or serial arrangement. Anaerobic digester units and methods for using digester units are known and may be incorporated in the methods and systems of the present invention. An anaerobic digester unit may an enclosed digester, which may be cylindrical, rectangular, square or irregularly shaped, may be plug flow, complete mixed, anaerobic granular sludge bed reactors, up flow anaerobic sludge blanket reactors, expanded granular sludge bed reactors, lagoon or covered lagoon, located above or in ground, with a cover of solid design, flexible membrane design or a combination of the two, which will capture and contain gaseous material produced by anaerobic digestion of the feedstock. The digester unit may be operated to maximize the digestion rate and gaseous material production by the primarily anaerobic bacteria, and to capture the gases for transfer to other components of the methods and systems of the present invention. These gases may include, but are not limited to, methane, butane, propane, carbon dioxide, oxygen, hydrogen sulfide, nitrogen containing gases, such as ammonia (NH₃) and others, may be affected by the specific makeup and source of the feedstock, such as from the input feeds and supplements ingested by the animals or plants providing the feedstock.

A design of an anaerobic digester unit may allow for the suspension of solids within its body, to encourage maximum contact of the anaerobic microorganisms with the nutrient rich components of the feedstock. The mechanisms for maintaining this suspension may be a mechanical mixing system of paddles, pumps, venturi induced jets, rotating discs or combinations of these. The agitation may be gentle in nature so as to avoid shear, cavitation, depressurization or overpressures which could harm the bacteria, interrupt reproduction of the bacteria, production of the output gases and/or digestion of the feedstock. During digestion, the volume of suspended solids will be reduced, such as through digestion, by amounts such as 5% to 25%, depending on the input material source, its nutrient values, the amount of foreign material included in the input material, residence time and the combination of anaerobes present.

Environmental parameters such as pH, volatile acid concentration, temperature, nutrient availability, heating and heat balance may influence the ability of the bacteria to produce optimum quantities of gas. These parameters are controlled and monitored to produce the optimum conditions for the fermentation cycle. For example, temperature ranges for optimum conditions for bacterial growth in the anaerobic digestion process include a mesophilic range, (85° F.-110° F.) and a thermophilic range, (110° F.-150° F.). Higher and lower ranges are also possible, depending on the bacteria used. Heat or energy to maintain the anaerobic digester unit in the desired temperature ranges may be provided from outside sources, or may be provided from transfer from the energy conversion unit or other units within the system.

The anaerobic microbes used in an anaerobic digester unit may be any anaerobic bacterium, fungus, mold or algae, or progeny or recombinant microbe thereof, which is capable of converting feedstock to a useful material in an anaerobic digester unit of the invention. Such anaerobes may be combinations of obligate anaerobic bacteria and facultative bacteria. Anaerobic microbes may be isolated from decaying or composted feedstock, may be endogenous to the area in which the feedstock was first obtained, may be obtained from bacterial or fungal collections such as those of the American Type Culture Collection (ATCC) or may have been genetically altered or engineered to convert a feedstock to a useful material. Anaerobic microbes that convert a cellulose-containing feedstock into methane, a nitrogen rich fertilizer, charcoal, humus or an insecticidal slurry are contemplated by the present invention. The anaerobic microbe may be a psychrophile, mesophile or thermophile. Generally, a mesophile will prefer operating temperatures in the range of about 60° 120° F., and a thermophile will prefer operating temperatures in the range of about 120°-160° F.

Examples of an anaerobic microbe which is useful in the anaerobic digester unit of the invention include yeast, a methanogenic bacterium, methanobacterium, acetobacterium, acetogenic bacterium, liquefaction bacterium, Clostridium spp. (methane), Bacillus spp., Escherichia spp., Staphylococcus spp., Methanobacter spp., Methanobacter (Mb.) omlianskii (methane), Mb. formicicum (methane), Mb. soehngenii (methane), Mb. thermoautrophicum (methane), Mb. ruminatium (methane), Mb. mobile (methane), Mb. methanica (methane), Methanococcus (Mc.) mazei (methane), Mc. vannielii (methane), Ms. mazei (methane), Mb. suboxydans (methane), Mb. propionicum (methane), Methanosarcina (Ms.) bovekeri (methane), Ms. methanica (methane), Ms. alcaliphilum (methane), Ms. acetivorans (methane), Ms. thermophilia (methane), Ms. barkeri (methane), Ms. vacuolata (methane), Propionibacterium acidi-propionici (methane), Saccharomyces cerevisae (ethanol), S. ellipsoideus (ethanol), Clostridium propionicum (propanol), Clostridium saccharoacetoper-butylicum (butanol), Clostridium butyricum (hydrogen), wherein the chemical in parentheses indicates a useful material which that microbe produces.

Other microbes and/or enzymatic catalysts can be added to the anaerobic digester unit to facilitate breakdown of the feedstock into components which are usable by the anaerobic microbe as either nutrients or starting materials for useful materials made by the anaerobic microbe. Such other microbes and/or enzymes include, but are not limited to, amylases, proteases, cellulases, hydrolases, lipid hydrolyzing enzymes, lysozymes, phosphatases, esterases, amidases, and lipases.

The conditions inside the anaerobic digester unit will vary according to the useful material being produced, the anaerobic microbe being used, the configuration of the anaerobic digester unit, the feedstock being converted, the desired productivity of the anaerobic digester unit, and the form of microbe (immobilized or free-flowing) used. Immobilized microbes can be prepared using any methods known by the artisan of ordinary in the arts. The conditions used to culture the anaerobic microbe and maintain it viable in the anaerobic digester unit can be varied. Conditions which can be controlled include solids content, reaction solution composition, temperature, gas content, digestion rate, anaerobic microbe content, agitation, feed and effluent rates, gas production rate, carbon/nitrogen ratio of the feedstock, pressure, pH, and retention time in the digester, among other things. The amount of solids in the digester unit may range from about 1 to about 60% wt., from about 20 to about 50% wt., or from about 40 to 50% wt. based upon the total solution weight. The particle size of solids in the digester unit may affect the rate of digestion. Generally, the smaller the particle size, the faster the rate of digestion.

The temperature of the reaction solution is generally in the range of about 60° F. to about 160° F., about 90° F. to about 118° F., about 90° F. to about 115° F., about 90° F. to about 110° F., or about 90°-95° F. The optimum operating temperature will depend upon the anaerobic microbe used, the product being produced, the pressure under which the digestion is conducted, the carbon to nitrogen ratio of the feedstock, and/or the contents of the feedstock. For Clostridium spp., a temperature is in the range of about 70°-100° F., about 70°-95° F., or about 75-95° F. For a mesophilic microbe, a level of productivity and purity methane is generally attained at a temperature in the range of about 90°-118° F.

When the digester unit is operated, the type of product gas formed may depend upon the operating pressure of the digester and the components of the discharge gas treatment system used to process and purify the gas. For example, the digester unit can be used to produce predominantly methane or carbon dioxide. Higher pressure generally promotes the conversion of carbon dioxide to methane and lower pressure generally leads to the formation of more carbon dioxide and less methane. Hydrogen sulfide may also be produced in the anaerobic digester unit. Nitrogen containing gases, such as ammonia, may be produced in an anaerobic digester unit. For example, ammonia may be separated from the gases produced in the anaerobic digester unit and used as a gas to be provided to another unit of the system, such as the algae production unit, or liquefied or added to other components to make fertilizers, or transferred to external sites to provide a nitrogen source for agriculture, landscaping or aquaculture.

Accordingly, when the optimal operating temperature for a particular combination of anaerobic microbe and feedstock slurry components is identified, the composition of the biogas produced can be altered by changing the pressure at which the digestion is conducted. Residence time in and size of the anaerobic digester unit may be dependent on input feedstock, added materials to the digestion fermentation, temperatures, atmospheric conditions in the area, altitude, and space limitations of individual sites.

The gases produced in the anaerobic digester unit are removed from the interior of the digester, such as from between the surface of the slurry and the roof, and piped or transported by other conduits, to a gas conditioning unit, in which the gases may be separated, if needed, into those which are combustible to yield energy, and those that interfere with combustion and energy yield. This separation may be accomplished by membranes, catalytic scrubbers or moisture removal techniques, or known methods for gas separation. The gases that are combustible are stored, transported to other locations or transferred to an energy generation unit (EGU). Those that are not combustible are stored, transported to other locations or transferred to another unit within the system for use in that unit. When the fermentation cycle, the digestion cycle, is completed, the solids and liquids of the anaerobic digester unit are removed from the digester and transported to another location, stored or transported to another component of the system. For example, the resultant solids and liquids of the anaerobic digester unit may be transported to an aerobic digester unit. The transfer of the resultant solids and liquid components from the anaerobic unit may be accomplished by means of a mechanical trash pump, disc pump, gravity flow piping, screw conveyor, belt conveyor, peristaltic pump, and/or combinations of these. Embodiments of the present invention may comprise transport of some or all of the solids and liquids resulting from anaerobic digestion which may be stored, transported to other sites, used as is, for example as fertilizers, or dried or concentrated. The byproducts of anaerobic digestion include solids, liquids and gases. Each of these may be conveyed to another unit or method step in the methods and systems of the present invention.

In an embodiment of the present invention, the solids and liquids resulting from anaerobic digestion are transported to one or more aerobic digester units, which may be used in serial or parallel arrangement. An aerobic digester unit may comprise a vessel of round, square, rectangular or combinations of these shapes which can be above ground and/or partially or completely below ground. A digester may be enclosed by a solid roof, a membrane suspended over the digester, or combinations of both, in order to capture for use in subsequent steps of the process, the gases produced in the aerobic digestion of the resulting solids and liquids from the anaerobic digester unit. These gases and water are conveyed, such as by pipes or conduits to other units of the system or method, or to other locations, or are stored. The aerobic unit may have an agitation design to maintain suspension of the solid materials within the aqueous matrix of the digester, and may incorporate an air/oxygen injection/induction system to provide adequate oxygen supply to the bacteria within the digester so as to maintain maximum digestion of the input material by aerobic means. An aerobic unit contains aerobic and facultative microorganisms that digest the input material, which may be in a slurry form from an anaerobic digester unit, so as to reduce the volume of material, release components from the solid and liquid material so the components may be more readily utilized, and to produce gases, including carbon dioxide, and help condition water. These aerobic output materials, include components from the solids and liquids, the remaining solids and liquids, gases produced by the aerobic microorganisms, and water. The aerobic output materials may be transported to another location, stored or used in other units or methods of the present invention.

Basic operation of an aerobic digester unit may be as follows. A pump, driven by a motor, conveys the liquids and solids, such as those resulting from the anaerobic digester unit, into an aerobic digester unit. The material in the aerobic unit is completely mixed, such as by the action of blades, paddles, pumps, sparging of oxygen or atmospheric air into the mixture, or venturis, and the agitation continues until whole digestion process is completed. Foaming may occur and may or may not be controlled by known methods. The gases produced are trapped and removed as aerobic digestion continues.

Aerobic digestion requires oxygen and, usually, aeration by atmospheric air is performed. However, since atmospheric air has an oxygen concentration of about 21 volume %, the aeration efficiency is low, in addition to a disadvantage of a higher dissipation rate of heat. It may be considered favorable to use a gas rich in oxygen, in particular, pure oxygen, in view of expenses for the heat insulation and for the aeration operation. However, use of an oxygen-rich gas suffers from a problem that exhaustion of carbon dioxide gas formed during the digestion becomes difficult, resulting in a decrease in the digestion activity. In order to warrant a flow rate of the aeration gas capable of expelling carbon dioxide gas formed during the digestion, it may be preferable to perform the aeration by supplying, in addition to the oxygen-rich gas, an amount of air required for discharging the carbon dioxide gas. As the oxygen-containing gas, there may be employed an oxygen-added air in which the concentration of oxygen in the atmospheric air is increased. The oxygen concentration in the oxygen-containing gas may favorably be at least 50% by volume, preferably at least 70% by volume. The pH may be controlled by the rate of addition of air so as to maintain the pH value of the digesting materials in the range from 5 to 9, from 6 to 8, or from 6.5 to 7.7.

One of the major differences between aerobic and anaerobic digestion is in the metabolic characteristics of the respective microorganism populations. In aerobic digestion, aerobic and facultative microorganisms use oxygen and obtain energy from the available biodegradable organic matter. The end products of aerobic digestion typically are carbon dioxide, water and materials such as polysaccharides, hemicellulose, and cellulose. During anaerobic digestion, facultative, obligate and strict anaerobes simultaneously hydrolyze complex organics and assimilate intermediate volatile organic acids. The end products of anaerobic digestion primarily are carbon dioxide, methane, and water. Aerobic organisms include those found in the biomass feedstock, and may include other microorganisms or larger organisms that are helpful in aerobic digestion.

The output material, which is a byproduct of the aerobic digester unit, comprises liquid and suspended solids, carbon dioxide and other gases. The solid and liquid output material from the aerobic digester material may be discharged as feedstock into the algae production unit, using mechanical pumps, gravity flow methods, process injection and metering systems, screw conveyors, peristaltic pumps, gaseous injection devices and combinations of these. The liquid and solid components of the output may be metered into the algae production unit (APU) wherein the aerobic digester output material may be used for production of gases, and algae production. The gas output material from the aerobic digester step may be removed from the interior of the digester and piped or transported by other conduits, to a gas conditioning unit, in which the gases may be separated, if needed, into those which are combustible to yield energy, and those that interfere with combustion and energy yield. This separation may be accomplished by membranes, catalytic scrubbers or moisture removal techniques, or known methods for gas separation. The gases that are combustible are stored, transported to other locations or transferred to an energy generation unit (EGU). Those that are not combustible are stored, transported to other locations or transferred to another unit within the system for use in that unit.

The combustible gases produced in the present methods and systems are burned in one or more energy generation units (EGU). Gases may be injected into an EGU, using internal combustion engines, fuel cells, turbo fan jet engines, and/or combinations of these which may be adapted for combustion of gases. Output from these engines may be used to power generators to produce electricity. Other components include, but are not limited to, phase converters, voltage converters and line conditioners. The byproduct gases produced during combustion may be captured, piped and utilized in other units or methods of the present invention.

Heat, a natural byproduct of combustion and energy generation, may be captured in a heat recovery unit, transferred to water to generate hot water, utilized in other units or methods of the present invention, or transferred to other external users of heat energy. These external users of heat include, but are not limited to, dairies, poultry producers, swine production units, or other industrial users of hot water energy. The electricity produced may be used on site, or sold to external customers, including nearby agricultural entities, public utilities desiring to purchase electricity produced by green methods, nearby industrial customers, or nearby residential subdivisions wishing to minimize their impact on the earth's environment and utilize green energy sources. Heat may also be used in units of the invention, such as an anaerobic digester unit, an aerobic digester unit, and/or the algae production unit. The heat may be used in one or more of these units to maintain an optimal temperature range for the organisms therein. For example, the heat from the energy production unit or a heat recovery module may be transferred to the algae production unit to augment the heat from solar sources to maintain an optimum temperature range for the algae growing therein. Additionally, heat may be transferred and used as a heat source for drying the algae in the algae harvesting unit, for example, with vacuum or spray drying processes.

Byproducts of the aerobic digester unit, such as solids, liquids or gases, may be conveyed to an algae production unit (APU). An APU is one unit where byproduct carbon dioxide from the electrical generation unit and/or output gases from the anaerobic digester unit and/or aerobic digester unit may be utilized in the production of algae. Algae, as used herein, includes eukaryotic and prokaryotic organisms, includes single-cell and multi-cell organisms, phytoplankton, and particular algae, include, but are not limited to, diatoms, Spirulina sp Scenedesmus acutus, Spirulina maxima, Cosmarium turpinii, Spirulina platensis, Chlorella sp., Spirulina maxim, Spirulina platensis, Chlorella ellipsoidea, Scenedesmus sp., Actidesmium, Albrightia, Amphitrrix, Anabaena, Ankistrodesmus, Aphanocapsa, Asterococcus, Calothrix, Chaetoceros, Chlodatella, Chlorococcum, Chlorophyta, Chodatella, Chrooccoccales, Chroococcus, Chroododdoles, Chrysophyta, Chrysostephanosphaera, Closterium, Coelosphaerium, Colteronema, Cosmarium, Crucigenia, Dimorphococcus, Euastrum, Eucapsis, Eugenoids, Eupheroids, Franceia, Scenedesmus, Gloeocapsa, Gloeotaenium, Golenkinia, Gomphosphaeria, Gonium, Holopedium, Hydrosera, Lagerheimia, Lyngbya, Merismopedia, Microsystis, Navicula, Oocystis, Oscillatoria, Palmella, Pediastrum, Pinnularia Chrysocapsa, Platydorin, Plectonema, Scenedesmus, Schizogonium, Selenastrum, Sorastrum, Staurastrum, Stauroneis, Tetrastrum, Trichodesmium, Trochiscia, Westella, Zygnema blue-green algae, photosynthetic bacteria such as Rhodospirillum sp. or Rhodopseudomonas sp. The algae convert the carbon dioxide and/or ammonia and other gases provided, nitrogen, phosphorous, potassium and other nutrients and minerals from other units of the system, such as the EGU, the anaerobic and/or aerobic digester units, into carbohydrates, proteins, lipids, fatty acids, enzymes and other beneficial products. The algae may also clean the water from the input slurries of suspended materials so that the water is usable in other units of the system or for external uses.

The carbon dioxide or other gases may be injected into the algae production unit via injection methods, including but not limited to, direct flow injection, air sparging, microbubble tubing, air induced venturi devices and/or rotating disc dispersion systems. The gas induction method aids in dispersion of the input gases into the aqueous matrix of the APU, and augments movement, stirring and mixing, and exposure of the algae to sunlight. Within the APU, there are several variables that contribute to the breakdown of the input components into a usable final product, including the water, effluent nutrients from the aerobic digester unit, the gas byproducts of the microbial digester units, and the EGU. Variables considered in the growth and maintenance of the algae culture, in addition to maximizing byproducts from the algae and water treatment aspects, include pH, temperature, sunlight concentration, carbon dioxide concentration, salinity, concentration levels of nitrogen, phosphorous and potassium, as well as concentrations and form of other minerals and organic materials. Methods of control, adjustment and conversion of input materials, along with methods of controlling the specific species of algae and related plant organisms are useful in maximizing the production of the desired algae, cleansing of the water and the oxygen release of the APU. The APU mixes input materials as completely as practical, and exposes the algae in the aqueous stage, to sunlight. This exposure allows the algae and other organisms to use photosynthesis to capture the energy from sunlight and facilitate the conversion of carbon dioxide to carbohydrates, proteins, fatty acids, lipids and other compounds, while cleansing the water and releasing oxygen to the atmosphere.

Oxygen is produced as a by-product of the photosynthetic activity of the algae and may also be produced in the aerobic digester unit. The oxygen can be released into the atmosphere to improve ambient oxygen levels, or may be captured and transported to an aerobic digester unit, an algae production unit, or to an energy generation unit. Providing oxygen to photosynthetic organisms or aerobic organisms enhances their activity and may reduce the residence time of the biomaterial within the unit. Providing oxygen to an energy production unit may increase the efficiency of the combustion reaction. The increased efficiency may result in the use of smaller energy generation units and may increase the percentage of conversion of the carbon of combustible input materials to CO₂. The increased CO₂ may be used in an algae production unit to increase its efficiency.

Additive materials may be added at any step in the method or to any units in the system where desired, for example to enhance growth of microorganisms, or to enhance the byproduct products. For example, a blend of extracted minerals, as described in U.S. patent application Ser. No. 10/725,729, may be added to enhance both the quantity and quality of algae produced, the remediation capability of the bacteria, and the quality of output materials from the algae. This extracted mineral composition can be introduced into the method or system of the present invention at multiple points in the process, or in pre-method and pre-system steps. These include, but are not limited to, ingestion by the animals which are the source of the biomass, at the aerobic and anaerobic digester units, in the APU and combinations of each of these.

The design for the APU may be dependent on external variables, such as the space available and the weather or temperature limitations of the site. APU designs include open-topped serpentine ponds, covered ponds, rectangular or square pools, clear tubing in serpentine flow patterns, clear tubing in linear layout, falling film, fixed film, triangle conduit, octagonal glass fixture and others of current design.

The harvesting process for recovery of algae from the APU involves processes which are implemented at the point at which the concentrations of the algae are adequate to be practical. The liquid in which the nutrient components, algae and byproduct solids are suspended is primarily water, and this liquid is drawn off of the APU using gravity flow, vacuum, pumping and/or conveyor to feed the slurry to a separation process which utilizes methods and apparatus, including screens, membranes, centrifuges, or cyclones. This process step concentrates and sequesters the algae and any solid byproduct materials into a vessel storage area, while separating the liquid and gaseous components of the slurry to be sent to other units or sequestered for use as an end product. The water component is then available for conveyance to external sources such as nearby livestock producers, light commercial entities, nearby agricultural entities for use in irrigation of crops, for irrigation of nearby recreational facilities, or as input water into a domestic water treatment plant for human use, conveyance internally in the methods and system units of the present invention, such as injection as feed water in at the debris separation unit.

From the harvest of algae grown in the APU, depending on the algae grown, multiple processes and products will result in many end products. The processes utilized will vary, depending on the input species of algae, the development stage the algae are in at the time of processing, the end product desired, and the specifics of the final product. For example, some algae produce oil naturally. These algae are processed to make biocrude oil and products that are refined to make gasoline, diesel, jet fuel, and chemical feedstocks for plastics and drugs. The algae and chlorophyll-containing organisms are a family of over 20,000 different known species. Over 5000 species of red algae, over 2,000 brown algae, and over 8,000 green algae, and over 650 fresh water and seaweeds have been reported. Commercial uses for algae include fertilizer runoff reclamation, sewage treatment, CO₂ sequestration, pollution control, animal feedstock, human nutritional supplementation, dyes and colorants, cosmetic and personal care, pharmaceuticals and bioplastics.

An example of the post harvest process comprises taking the dewatered algae mass and further drying it using a heated drum dryer, a spray dryer, heat augmented vacuum drying, or other equipment to dry the algae to a dry powder form. Depending on the characteristics of the end product, these algae may be ground to an ultra fine powder for inclusion in food products for human consumption, animal feed, input for organic fertilizers, input for cosmetic products or as a source for pharmaceutical products to be developed outside this facility. Alternately, the dewatered, but as yet wet form of algae may be further processed to extract various oils, lipids, fatty acids and enzymes from the algae mass, depending on the final product(s), and the species of algae grown.

Various forms of extraction methods for extracting products from the algae are contemplated in the present invention, including but not limited to, mechanical crushing, supercritical fluid/CO₂ extraction or chemical solvent extraction such as hexane, since many algae components are hydrophobic, enzymatic extraction, osmotic shock extraction, and ultrasonic extraction. The CO₂ produced in the methods and systems of the present invention may be used in CO₂ supercritical methods of extraction of the algae. The oils, lipids, fatty acids, and enzymes extracted from the algae can be used in many commercial products or methods, including but not limited to, cosmetics, nutritional purposes, and pharmaceuticals. For example, some algae are a good source of vitamins, including vitamins A, B1, B2, B6, niacin, and C. As another example, Sprirulina (Arthrospira platensis) is useful in dietary supplements and for other nutritional supplementation purposes. It is a blue-green algae (cyanobacterium) and provides nutritive value due to its high amount of protein and the oils it produces such as omega-3 and omega-6, docosahexanoic acids (DHA) and eicosapentaenoic acids (EPA) fatty acids. Such fatty acids are typically found in fish and have been clinically proven to provide health benefits. Spirulina as well as other types of algae may serve as both a food source and an air purification system. Because the algae are a sources of nutrients, algae can serve as animal feed, for example for poultry and cattle, as well as used to fertilize crops. Algae may also be used to remove lead and other contaminants from polluted water. Commercially pure spirulina and other algae provide coloring agents that are used in manufacturing and pharmaceuticals. The major carotenoids of Spirulina are carotene, cryptoxanthin and zeaxanthin. Additionally other colorants can be extracted from algae, such as a red carotenoid, astaxanthin, derived from the microalgae Haematococcus pluvialis. Spirolina may also be added to cosmetics to provide antioxidant properties and may also have potential anti-aging effects. Algae may be added to foods or chemicals as a preservative.

Different types of algae may provide specific extracted products. For example, Parietochloris incise (a green algae) is found to have high amounts of arachidonic acid. Chlorella and Dunaliella have been found to have high amounts of carotinoids. Prebiotic ingredients that boost the growth of beneficial bacteria in the gut such as algae-derived oligosaccharides have been reported to have benefits. Polysaccharides from brown marine algae may be used as ingredients for food and beverages for example, by extracting laminarans and fucoidans from brown algae, which may hsbr immunostimulant, anti-viral and anti-cancer properties.

Algae have been shown to have pharmaceutical activities such as anticoagulants, antibiotics, antihypertensive agents, blood cholesterol reducers, dilatory agents, insecticides, and anti-tumorigenic agents. Cyanobacteria (blue-green algae) provide compounds such as antibiotics and other pharmacologically active compounds. Algae may provide other beneficial compounds with novel structures which are yet to be isolated and characterized, all of which may have multiple applications in many areas including medicine, agriculture, nutrition, and cosmetics.

In cosmetics, algae may provide compounds for or be used as thickening agents, water-binding agents to serve as moisturizers, and for antioxidant effect. For example, algae such as Irish moss and carrageenan, contain proteins, vitamin A, sugar, starch, vitamin B1, iron, sodium, phosphorus, magnesium, copper, and calcium and are used in cosmetic preparations for their conditioning effect on skin. Blue green algae can also be used as biofertilizers. Such fertilizers are more complex than chemical fertilizers because the algae have the ability to carry out both photosynthesis as well as nitrogen fixation.

Another use of the algae produced is that the lipids and fatty acids produced by specific species of algae can be processed to yield biodiesel, ethanol, methanol, and alcohols, and other energy sources for transportation, agriculture and aviation users, or burned to produce energy and byproduct gases and heat, that can be used in the systems and methods of the present invention, or provided to external sources.

Output materials such as gases, may be produced in units such as an hydrolysis unit, an anaerobic digester unit, an aerobic digester unit, an algae production unit and/or an energy generation unit. The gases are collected from the unit in which they are produced by conduits or pipes and may be transported to a gas separation unit, where the different gases are separated by methods known to those skilled in the art. The separated gases, for example, oxygen, hydrogen, ammonia, carbon dioxide, hydrogen sulfide, sulfur containing gases, nitrogen containing gases, carbon monoxide, hydrogen containing gases, carbon containing gases, may be burned for energy production, may be provided to a unit of the present invention as an input material, may be stored as a gas, liquid or solid, may be transported as a gas, liquid or solid to sites external to the units of the present invention for other uses.

In FIG. 1, an exemplary method and system is provided. Biomass 1 is provided to debris separation unit, 2, for separation of oversized debris 2a. Biomass 1 may be suspended in water 11 such as water removed from other steps of the method or units, such as from algae harvesting. The biomass separated from the oversized debris, biomass feedstock 2b, is then conveyed to an hydrolysis unit 3, where gases 3a, such as carbon dioxide are removed and provided to other units, such as an algae production unit (not shown). The resulting biomass feedstock 3b is transported to an anaerobic digester unit 4. The biomass feedstock 3b is acted on in the anaerobic digester unit 4 by the microorganisms therein. Heat 12 may be provided from a heat recovery unit 9. Gases 4a produced in the anaerobic digester unit 4, such as methane, carbon dioxide and ammonia, are removed to a gas separation unit 10 and combustible gases 10b are separated and removed to an energy generation unit 8. Gases which are useful in other units of the system, 10ab, such as ammonia, are transported to one or more units. The resulting solids and liquids in the anaerobic digester unit 4, 4b, are removed and transferred to an aerobic digester unit 5.

In the aerobic digester unit 5, input gases 5d may be added, which may come from the gas separation unit 10 or from external sources. The gases 5a/5b from aerobic digester unit 5, such as ammonia and carbon dioxide, are removed and transferred to the gas separation unit 10 (5a) or may be transferred to the algae production unit 6 (5b). The solids and liquids resulting from aerobic digester unit 5, 5c, may be dried or have water added or may be separated into liquid components or solid components, or any other components or the whole mixture of solids and liquids may be transferred to the algae production unit 6. Elements may be added to the units from external sources. For example, compressed air or other gases may be provided to a unit, as shown here, compressed gas, 5d, is pumped into the aerobic digester unit.

In the algae production unit 6, input gases are provided, 5b, 10a, 8a, such as carbon dioxide, ammonia, and the gases may be provided by other units of the system, such as the gas separation unit 10 or the energy generation unit 8. After sufficient growth of the algae and/or production of by-products by the algae, the algae and any byproducts 6b are harvested in the algae harvesting unit 7. Water 11 is removed and transferred to external sites or to other sites within the system. The algae and/or its byproducts may be further processed 7b.

The gases produced in units in the system may be transferred to energy generation unit 8, and if needed, are first treated by separation of combustible gases in the gas separation unit 10, and the combustible gases, 10b, are transported to energy generation unit 8, and gases that can be used in other units, 10a, are transported to one or more units. FIG. 1 shows usable gases 10a begin transported to algae production unit 6. Energy generation unit 8 generates electricity by burning the combustible gases to produce electricity 8c, heat 12 and output gases 8a, each of which may be transferred to other units within the system or provided to external sites. The heat recovery unit 9 may store the heat generated in forms such as hot water or hot air, and transfer it to other units in the system or to external recipients.

FIG. 2 shows an exemplary method and system of the present invention. Biomass 1 is provided to debris separation unit, 2, for separation of oversized debris 2a. Biomass 1 may be suspended in water 11 such as water removed from other steps of the method or units, such as from algae harvesting. The biomass separated from the oversized debris, biomass feedstock 2b, is then conveyed to an anaerobic digester unit 4. The biomass feedstock 2b is acted on in the anaerobic digester unit 4 by the microorganisms therein. Heat 12 may be provided from a heat recovery unit 9. Gases 4a produced in the anaerobic digester unit 4, such as methane, carbon dioxide and ammonia, are removed to a gas separation unit 10 and combustible gases 10b are separated and removed to an energy generation unit 8. Gases which are useful in other units of the system, 10a, such as ammonia, are transported to one or more units. The resulting solids and liquids in the anaerobic digester unit 4, 4b, are removed and transferred to an aerobic digester unit 5.

In the aerobic digester unit 5, input gases 5d may be added, which may come from the gas separation unit 10 or from external sources. The gases 5a/5b from aerobic digester unit 5, such as ammonia and carbon dioxide, are removed and transferred to the gas separation unit 10 (5a) or may be transferred to the algae production unit 6 (5b). The solids and liquids resulting from aerobic digester unit 5, 5c, may be dried or have water added or may be separated into liquid components or solid components, or any other components or the whole mixture of solids and liquids may be transferred to the algae production unit. 6.

In the algae production unit 6, input gases are provided, 5b, 10a, 8a, such as carbon dioxide, ammonia, and the gases may be provided by other units of the system, such as the gas separation unit 10 or the energy generation unit 8. After sufficient growth of the algae and/or production of by-products by the algae, algae and any byproducts 6b are harvested in the algae harvesting unit 7. Water 11 is removed and transferred to external sites or to other sites within the system. The algae and/or its byproducts may be further processed 7b.

The gases produced in units in the system may be transferred to energy generation unit 8, and if needed, are first treated by separation of combustible gases in the gas separation unit 10, and the combustible gases, 10b, are transported to energy generation unit 8, and gases that can be used in other units, 10a, are transported to one or more units. FIG. 2 shows usable gases 10a transported to algae production unit 6. Energy generation unit 8 generates electricity by burning the combustible gases to produce electricity 8c, heat 12 and output gases 8a, each of which may be transferred to other units within the system or provided to external sites. The heat recovery unit 9 may store the heat generated in forms such as hot water or hot air, and transfer it to other units in the system or to external recipients.

Methods of the present invention comprise treating biomass, comprising, providing biomass to one or more microbial digester units, which may an anaerobic digester unit and/or an aerobic digester unit, transferring output from one or more microbial digester units to at least one algae production unit, and harvesting the algae from the algae production unit, wherein gas(es) produced in at least one of the units is transferred to one or more of a gas separation unit, another digester unit, to a combustion unit or to an algae production unit. Heat produced in at least one of the units may be transferred to at least one of the units, and electricity produced in at least one of the units may be transferred to at least one of the units. The methods and systems comprise the equipment, such as conduits, pumps, valves, wires, switching equipment, necessary when transferring liquids, solids, gases, electricity and heat from one place to another. A method may comprise further retaining the biomass in at least one hydrolysis unit prior to providing the biomass to a microbial digester unit. A method may further comprise pretreating the biomass to remove debris prior to providing the biomass to an hydrolysis unit or a microbial digester unit. A method may further comprise extracting desired products from the harvested algae. A method may further comprise transferring heat or electricity produced in a unit of the method to one or more other units of the method. A method may further comprise using water from one unit in the operation of another unit or transferring the water to an external recipient, such as to agricultural uses or municipal water treatment plants. The algae produced is processed to produce biodiesel fuel, oils, lipids and fatty acids, protein for use in animal and human food, enzymes and alcohols for industrial uses and other beneficial products.
Systems comprise one or more hydrolysis units; one or more microbial digester units; at least one algae production unit; and gas transferring equipment comprising at least conduits and pumps. Systems further comprise at least algae harvesting units, or one or more debris separation units, heat transfer and electrical transfer conduits and pumps.

A method for producing energy and beneficial products from biomass, comprises removing large debris from biomass with a debris separation unit to results in a biomass feedstock; transferring the biomass feedstock to an hydrolysis unit and retaining the biomass feedstock therein for about 2 to about 48 hours; providing output from the hydrolysis unit to one or more anaerobic digester units; providing output from the one or more anaerobic digester units to one or more aerobic digester units; providing output from one or more aerobic digester units to one or more algae production units; harvesting algae from the algae production unit; wherein gases produced in at least one unit are transferred to one or more of a gas separation unit, another digester unit, to a combustion unit or to an algae production unit; and wherein energy produced in a combustion unit is transferred to one or more units; and wherein heat produced by one or more units is transferred to one or more units.

It must be noted that, as used in this specification and the appended claims, the singular forms “a,” “an,” and “the” include plural referents unless the context clearly dictates otherwise.

All patents, patent applications and references included herein are specifically incorporated by reference in their entireties.

It should be understood, of course, that the foregoing relates only to preferred embodiments of the present invention and that numerous modifications or alterations may be made therein without departing from the spirit and the scope of the invention as set forth in this disclosure.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

A system of the present invention is used to process and treat the biomass from 5,000 dairy cattle, or 12,500 hogs, or 1.2 million chickens. The biomass is pretreated to remove debris in a debris separation unit. Water is added to make the biomass feedstock a flowable mixture and the biomass is transported to the hydrolysis unit where it is retained for about 20 hours. More than one hydrolysis unit tank may be used for large amounts. Carbon dioxide gas is released in the hydrolysis unit, and that gas is pumped out of the hydrolysis unit container and the gas is provided to the algae production unit.

After about 20 hours, the biomass feedstock is pumped to an anaerobic digester unit, where the biomass is acted on by anaerobic microbes for about 3 days. Methane gas and ammonia gas, along with minor amounts of other gases, are produced and pumped to a gas separation unit. The gases are separated, and the methane is pumped to an energy production unit where it is burned in the process of producing electricity using a gas turbine. A megawatt of electricity is produced and a portion is used to run the pumps used in the system and to agitate the solutions in the microbial digester units. The ammonia gas is pumped to an algae production unit.

After about 3 days, the resulting liquids and solids from the anaerobic digester unit are pumped to an aerobic digester unit. To aid the activity of the aerobic microorganisms, compressed air is pumped into the aerobic digester unit. The biomass remains within the aerobic digester unit for about 5 days. Several gases are produced in the aerobic digester unit. The gases, such as ammonia and carbon dioxide are pumped to a gas separation unit, where the gases are separated, and the ammonia and carbon dioxide are provided to an algae production unit.

After about 5 days, the resulting liquids and solids from the aerobic digester unit are provided to an open pond of algae. In the open pond, 1.5 to 3 pound/square ft of algae are produced. In optimum conditions, 75 pound/sq ft is possible. The pond may be as large as 40 acres. The gases from other units are bubbled into the pond at various places in the pond. Heat may also be provided to the pond from other units. The algae grow. The activity of the algae remove nutrients and other elements from the water, so that the water, when separated from the algae, can be used safely for agriculture or to add to municipal water system treatment plants.

After about 2 weeks to 2 months of growth of the algae, the algae are harvested by draining the now-cleaned water from the pond and removing the algae with mechanical reapers. The algae are processed by pressing them to release biodiesel compounds. Alternatively, with other algae, the algae are dried and the dried material is used as animal feed.

What is claimed is:

1. A method for treating biomass, comprising,
a) providing biomass to one or more microbial digester units;
b) transferring output from one or more microbial digester units to at least one algae production units;
c) harvesting the algae from the algae production unit,
wherein gases produced in at least a) are transferred to one or more of a gas separation unit, another digester unit, to a combustion unit or to an algae production unit.

2. The method of claim 1, further comprising, retaining biomass in at least one hydrolysis unit prior to a).

3. The method of claim 1, further comprising pretreating biomass to remove debris prior to a).

4. The method of claim 2, further comprising pretreating biomass to remove debris transferring the biomass to an hydrolysis unit.

5. The method of claim 1, further comprising d) extracting desired products from the harvested algae.

6. The method of claim 1, further comprising transferring heat produced in a unit of the method to one or more other units of the method.

7. The method of claim 2, wherein the biomass feedstock is retained for about 2 to about 48 hours.

8. The method of claim 1, further comprising using the gas produced to generate electricity.

9. The method of claim 8, wherein electricity produced may be provided to one or more units of the method or to an external recipient.

10. The method of claim 1, further comprising providing the water from the algae production unit to another unit of the method or to an external recipient.

11. The method of claim 2, further comprising transferring gases produced in at least one hydrolysis unit to one or more of a gas separation unit, another digester unit, to a combustion unit or to an algae production unit.

12. The method of claim 1, wherein one or more microbial digester units is an anaerobic digester unit.

13. The method of claim 1, wherein one or more microbial digester units is an aerobic digester unit.

14. The method of claim 1, wherein biomass is provided to one or more anaerobic digester units and output from one or more anaerobic digester units is provided to one or more aerobic digester units.

15. The method of claim 1, further comprising,
d) processing the harvested algae to produce biodiesel fuel, oils, lipids and fatty acids, protein for use in animal and human food, enzymes and alcohols for industrial uses and other beneficial products.

16. A system for treating biomass, comprising,
a) one or more hydrolysis units;
b) one or more microbial digester units;
c) at least one algae production unit; and
d) gas transferring equipment comprising at least conduits and pumps.

17. The system of claim 16, further comprising, at least algae harvesting units.

18. The system of claim 1, further comprising a debris separation unit.

19. The system of claim 16, further comprising heat transfer and electrical transfer conduits and pumps.

20. A method for producing energy and beneficial products from biomass, comprising,
a) removing large debris from biomass with a debris separation unit to results in a biomass feedstock;
b) transferring the biomass feedstock to an hydrolysis unit and retaining the biomass feedstock therein for about 2 to about 48 hours;
c) providing output from the hydrolysis unit to one or more anaerobic digester units;
d) providing output from the one or more anaerobic digester units to one or more aerobic digester units;
e) providing output from one or more aerobic digester units to one or more algae production units;
f) harvesting algae from the algae production unit;
wherein gases produced in at least one unit are transferred to one or more of a gas separation unit, another digester unit, to a combustion unit or to an algae production unit; and wherein energy produced in a combustion unit is transferred to one or more units; and wherein heat produced by one or more units is transferred to one or more units.
